(19) United States
(12) Patent Application Publication
(10) Pub. No.: US 2022/0313876 A1
(43) Pub. Date: Oct. 6, 2022

(54) NURSING DEVICE AND STATION

(71) Applicant: MilkMate Products, LLC, Port Chester, NY (US)

(72) Inventors: Patrice Meagher, Port Chester, NY (US); Michael Meagher, Port Chester, NY (US); Alana Clerkin, Philadelphia, PA (US); Vladlena Belozerova, Philadelphia, PA (US); Richard T. Briganti, Bala Cynwyd, PA (US); Rui Yuan Chen, Philadelphia, PA (US); Joshua Funamura, Oakland, CA (US); Joseph Daniel Massam, Langhorne, PA (US); Meggie Oudheusen, Mount Vernon, NH (US); Maddy Ross, Newtown Square, PA (US); Matthew Selnick, Ambler, PA (US); Breanna Stachowski, Elma, NY (US); Sharon West, Elkins Park, PA (US)

(73) Assignee: MilkMate Products, LLC, Port Chester, NY (US)

(21) Appl. No.: 17/681,041

(22) Filed: Feb. 25, 2022

Related U.S. Application Data
(60) Provisional application No. 63/153,772, filed on Feb. 25, 2021.

(51) Int. Cl. A61M 1/06; A61M 1/00

(52) U.S. Cl. CPC A61M 1/062; A61M 1/604; A61M 1/74; A61M 2205/505; A61M 2205/3584

(57) ABSTRACT

A modular breast pumping station includes modular components that include: a multiuser breast pump configured to attach to disposable and recyclable tubing and breast shields; a storage area for the tubing and breast shields, and also containing storage bags for milk obtained from a user's breast; and a refrigerator for storage of the storage bags.

### BACKGROUND

Consider these statistics. 83% of mothers breastfeed and 90% of breastfeeding working mothers use a breast pump to store milk for their child. 50% of employers have a dedicated space for mothers to pump. And 60% of mothers stop breastfeeding before they would like to, citing as a common reason, “unsupportive work policies.” We are not doing a great job supporting these moms.

This, despite the known advantages of breastfeeding. According to the Cleveland Clinic, breastfed babies have stronger immune systems, less diarrhea, constipation, gastroenteritis, gastroesophageal reflux, and preterm necrotizing enterocolitis (NEC), fewer colds and respiratory illnesses like pneumonia, respiratory syncytial virus (RSV) and whooping cough, fewer ear infections, especially those that damage hearing, fewer case of bacterial meningitis, better vision and less retinopathy of prematurity, lower rates of infant mortality, lower rates of Sudden Infant Death Syndrome (SIDS), and less illness overall and less hospitalization.

The benefits are not solely with the baby either. Mothers benefit because breastfeeding promotes faster weight loss after birth, burning about 500 extra calories a day to build and maintain a milk supply; stimulates the uterus to contract and return to normal size; results in less postpartum bleeding; shows fewer urinary tract infections; decreases chance of anemia; and invites less risk of postpartum depression and more positive mood.

The long-term benefits to the baby and mother are numerous as well. The point being that breastfeeding is not just good for the mother and child, it's good for all of us.

In spite of this, we still shove the breastfeeding or pumping mother out of sight. Even in more progressive workplaces, the pumping mother will often end up in an unused room, which room is usually unused because it is undesirable. The mother gets a double-dose of feeling cast off in this pumping dungeon, often with nowhere to store her pumped milk except the office lunch refrigerator next to left over Italian subs and three-week-old macaroni. Shoving the breastfeeding or pumping mother out of sight is not necessarily a bad thing, since privacy is what she desires. We do humiliate her, however, by requiring she keep her pumping equipment at her work station, until the next time she has to carry it to the pumping dungeon past the prying eyes of male colleagues.

On top of this, in a best case, the mother in the pumping dungeon has perhaps lugged her heavy and expensive pump, with (she hopes) all attachments in her car or on the train, where it may get broken or contaminated, to the office. This is the pump that, whether she brought it from home or she keeps at the office, she has to carry through the office to the dungeon and disinfect in (usually) a publicly available restroom sink. If she's lucky, her employer provides the pump and this saves her money, but this does not avoid the disinfection issues, and it does not avoid sharing problems. Missing a feeding (or pumping session) produces painful engorgement, and signals the body to stop producing replenishment milk supply. A key point to stress here is that any pump that a mother would bring from home (or keep at the office) will require a half dozen or more components that must be assembled before each use, and then disassembled and washed afterwards. Obviously this is time-consuming and inefficient. But more importantly, if any of these parts are missing, the pump will not function properly and the user will have to forego the session or go buy or borrow a part (not ideal). Much efficiency is lost when pumping—time away from concluding work necessary for her job often causes her to have to work more time in the office or later at home when she could be spending time with her family.

The mom-on-the-go really has no options. If she needs to pump, the best she can hope for is an accessible private bathroom where she must pump amidst the refuse of whatever the public left behind before. Again, not only is the mom-on-the-go inconvenienced by lack of proper facilities, but she also had better have her pump and all its parts or even that public restroom does no good.

Pumping also poses issues for the employer or business owner where mothers wish to pump—retaining talent, success from mothers, morale becomes an issue when there is not a sufficient solution for the pumping mother. Employers desire to support and retain the working mothers among their staff, and many have moved to providing enhanced maternity benefits, including dedicated breast pumping rooms and even supplying these rooms with breast pumps. However, this does not solve the problem of the user being required to bring along her own pumping equipment or at the very least, personal kit of attachment parts.

The pumping station and disposable parts herein, which the inventors often call a pod or a cart, addresses these concerns and many more.

### SUMMARY OF THE EMBODIMENTS

A modular multiuser breast pumping station includes modular components that include: a breast pump configured to attach to disposable and recyclable tubing and breast shields; a storage area for such disposable and recyclable tubing and breast shields, and also containing empty milk storage bags; and a refrigerator for storage of the milk filled storage bag. This pod or cart allows for a mother to visit the pod without anything in order to pump (relieving the mother from the hassle and time associated with transporting and assembling and breaking down and cleaning pump and parts in order to pump)

### BRIEF DESCRIPTION OF THE DRAWINGS

The figures compliment the description herein.

FIGS. 1A-1C show different views of a breast pumping station as a kiosk.

FIGS. 1D-1F show a different kiosk variation.

FIGS. 2A and 2B show a breast pumping station as a cart.

FIG. 3 shows a combination of a breast pumping station as a cart and refrigerator.

FIGS. 4A-C show different packaging and designs for disposable and recyclable shields, diaphragms, tubing and storage bags.

FIGS. 5A-5E show different embodiments of the pump

FIG. 6 shows an embodiment of the packaging within the storage area.

FIGS. 7A and 7B show an application functionality map.

FIG. 8 shows an application virtual refrigerator.

### DETAILED DESCRIPTION OF THE EMBODIMENTS

The system herein includes some or all of an application that may be in interactive communication with an inventory tracking system and network of users and stations, as well as a nursing and breast pump station.

The Station

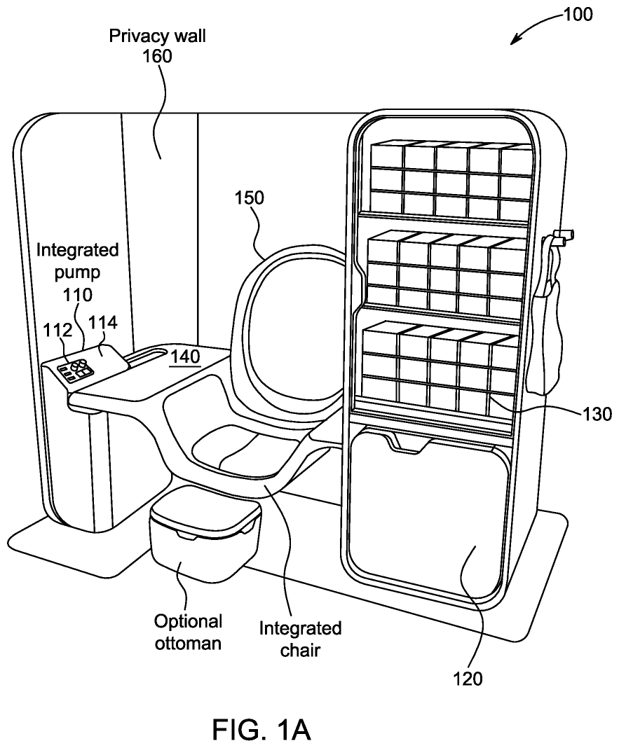

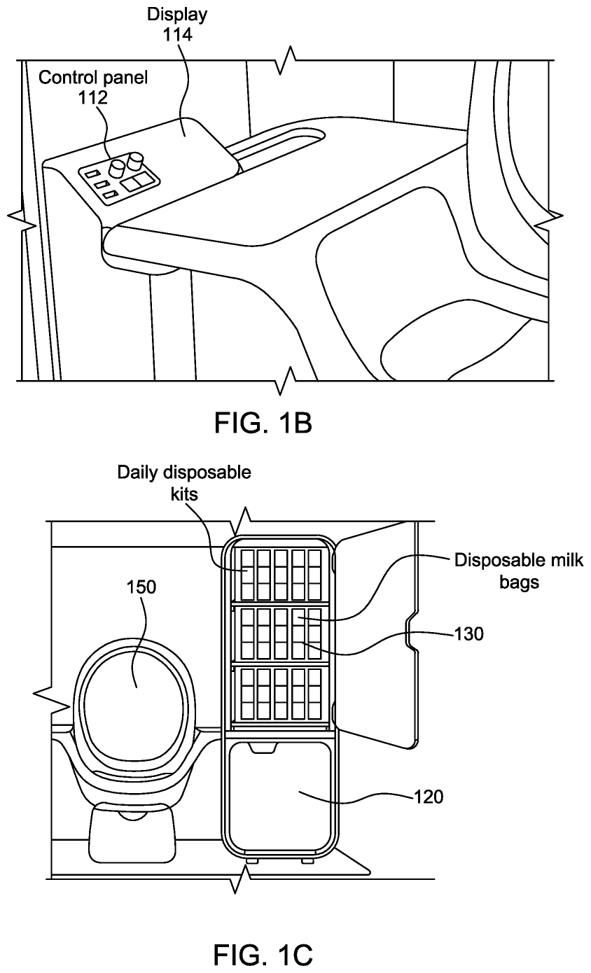

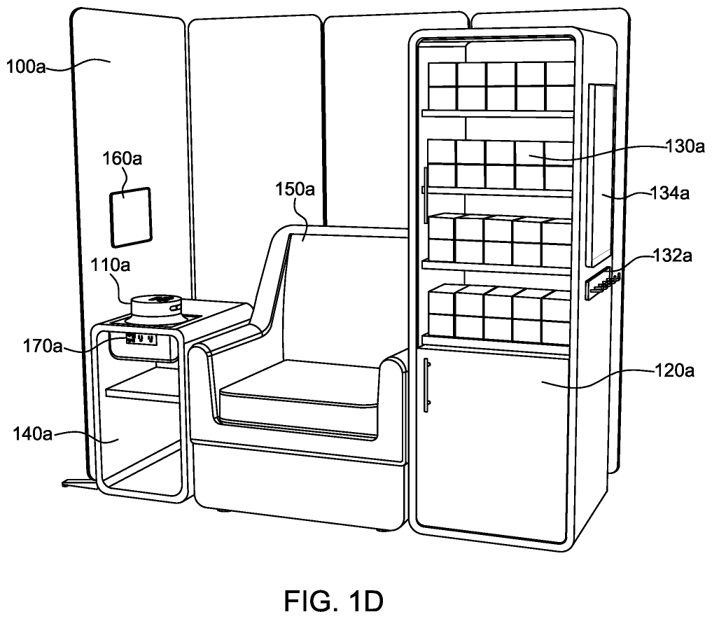

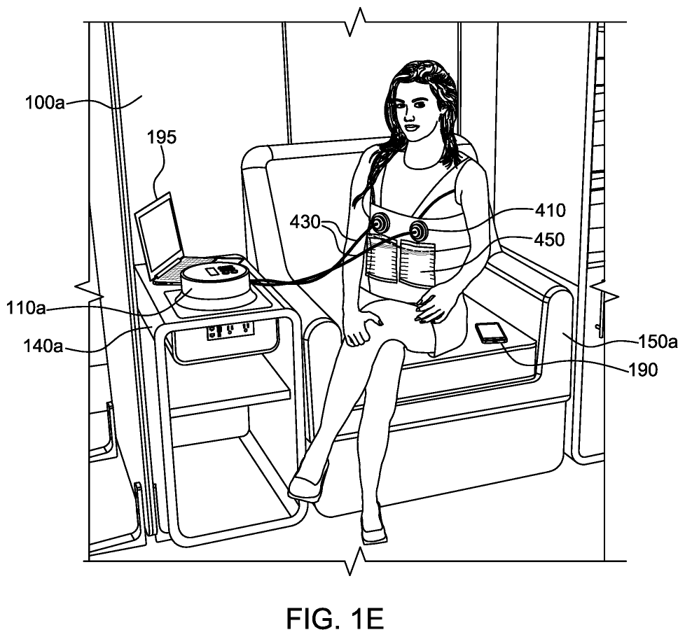

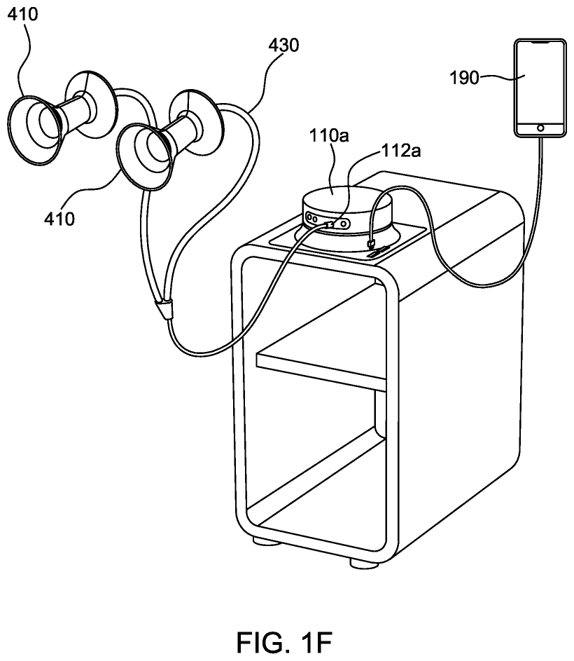

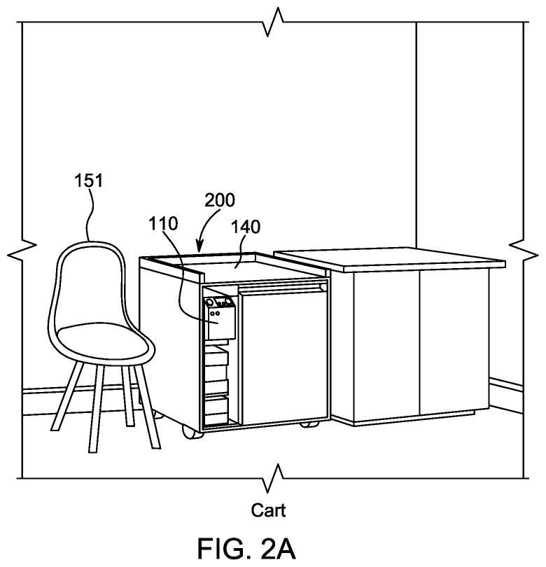

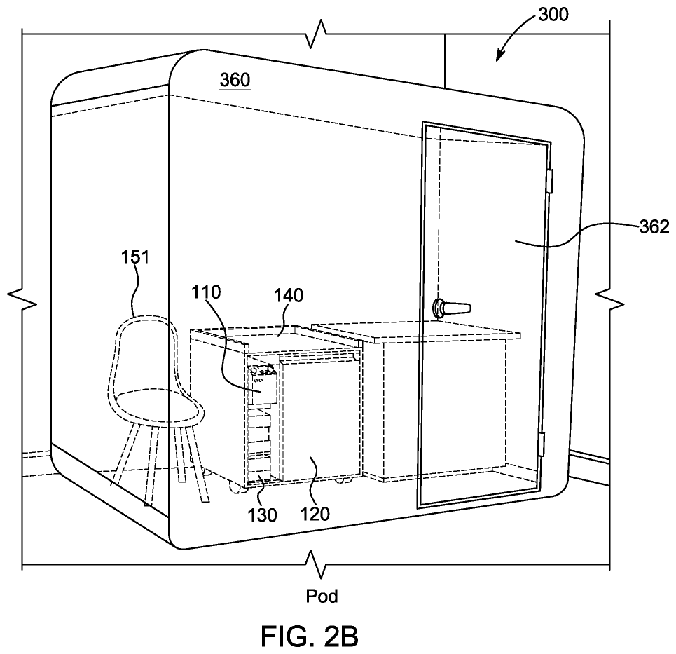

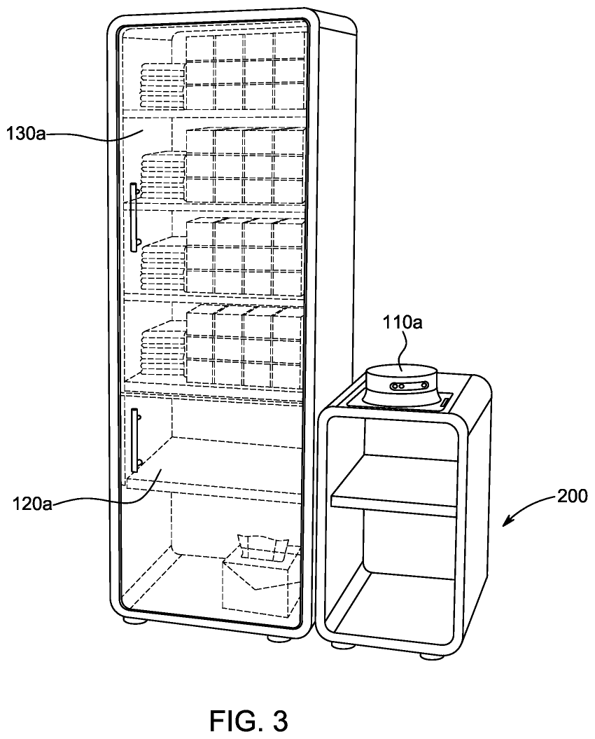

The station portion of the system includes a pod that works with a breast pump, as shown herein, and FIG. 3. The station may be a kiosk 100 (FIGS. 1A-1G), cart 200 (FIGS. 2 and 2A), or pod 300 (FIG. 3). All of these include certain similar elements, although for reasons of space limitations, they may be sized and arranged differently.

For example, the common elements may include a pump 110, refrigerator 120, supply storage area 130, and work surface/table 140.

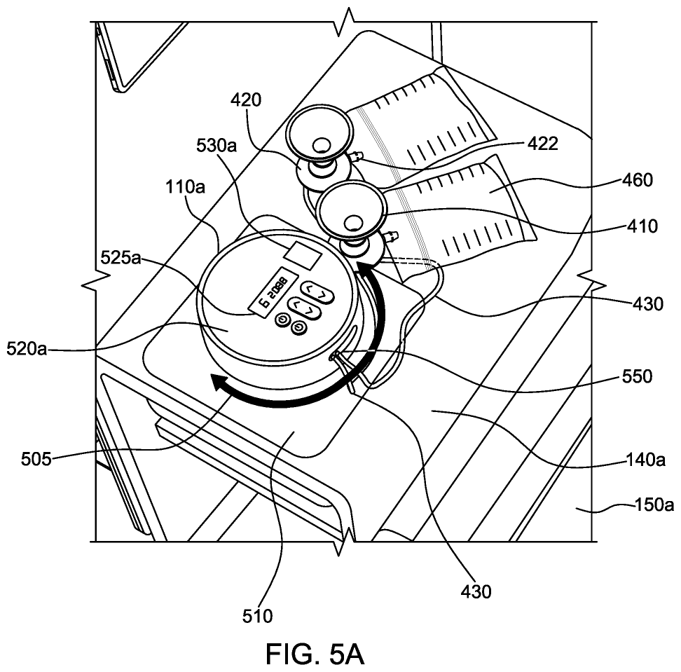

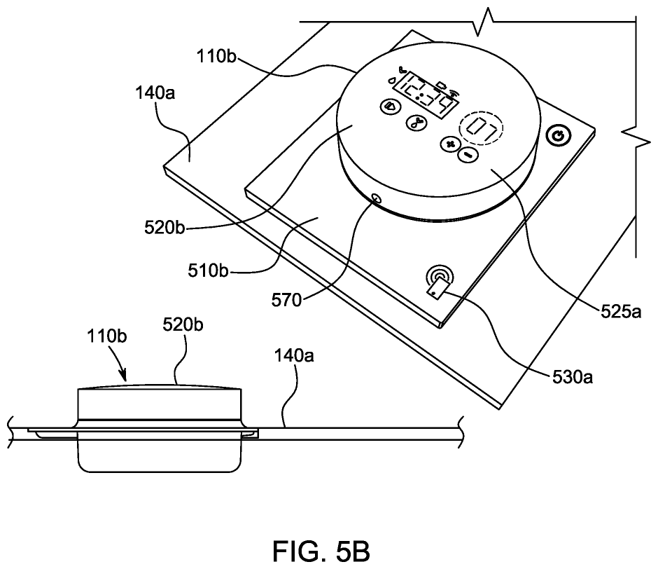

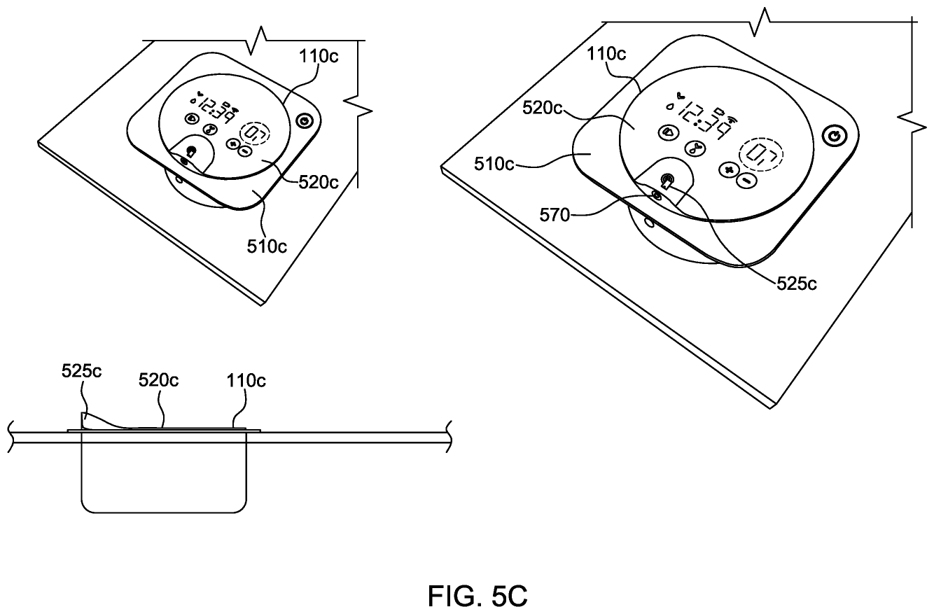

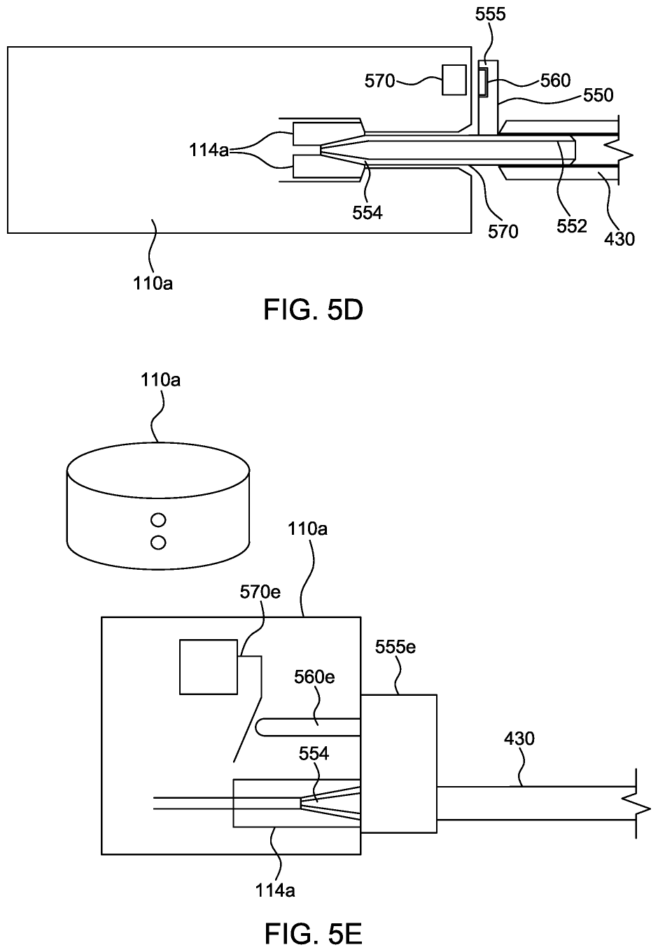

The pump 110, which will be described in more detail later with respect to FIGS. 5A-5E may be any pump that generates adequate vacuum to draw milk from the nursing mother and direct it to a storage receptacle 450 like a bottle or bag, and since it may be a permanent fixture of the system, it may include many other features. These may include: ability to store preset settings (vacuum strength, cycling, cycling strength, vacuum release, duration), vacuum level, vacuum cycle speed, mode: expression or stimulation (a.k.a. let-down), cycle active or cycle paused that a user may set and store or that may be accessible and operable through a mobile app that connects to the pump. The user settings may be stored at the pump 110 or in the mobile app that controls the pump 110. The pump 110 may include a control panel 112 and display 114 that control and display pump settings and information. The pump 110 ideally requires no or only surface cleaning by the user and/or property owner, and infrequent cleaning and updating by a qualified technician.

A storage area 130 may include disposable and/or recyclable tubes, milk storage bags, disposable cloths, disinfectant wipes, and breast shields (of a variety of sizes) necessary for pumping. A later section describes these accessories in detail but as supplies run low, the system may include a way to order resupplies. This may work through an inventory tracking system that works like a vending machine, individually tagged supplies (using RFID, NFC, barcode, etc), or just user input (a user may indicate when supplies are low). Inventory management may also be done using scales that send a signal when the weight on a shelf or hanger drops below a preset weight, indicating that it is time to order more supplies. All of these stored items may be disposable and single use only, which if properly sealed, guarantees their sterility.

The system may include a refrigerator 120 to store pumped milk. The refrigerator may include therein separate sections organized by name to avoid mixing up filled milk bags. Each section may be secured to the user only or just include identifiers. Further, the cart/kiosk/pod may be equipped with a label maker, printer or just labels to allow mothers to label their filled milk bags to avoid confusion.

The systems may also include a surface 140 upon which a user can place their belongings or work-related devices (inclusive of outlets for chargers, etc) and papers. The surface 140 may also be a convenient place to rest milk storage bags and other pumping-related supplies—or even the mother's own personal pump.

FIG. 1D shows an alternate embodiment of the above components, arranged in a different manner, that includes a kiosk 100a, seat 150a, ottoman (not shown), pump 110a, table 140a, storage unit 130a, and refrigerator 120a. In this embodiment of the pump 110a shown in FIGS. 5A-5H, the pump 110a includes inputs 112a that connect to tubing 430 and shields 410 discussed later. Further in this embodiment, the pump 110a may include a charger or controller input for a mobile phone 510 or may be controlled via wireless connection.

The kiosk 100a may also include a screen 160a that may show advertising or be connected to a smart device of the user, welcoming her to the kiosk 100a. The screen 160a may show content connected to the user via their smart device 510 or a login to the kiosk 100a, such content being directed advertising, instructions for use, images and videos selected by the user, or historical information.

FIG. 1E shows the kiosk 100a of FIG. 1D in use. As shown, the user will have removed a package 400 from the storage unit 130a and assembled it by attaching the tubing 430 between the pump 110a and diaphragm 420, which is in turn attached to (or integral with) the breast shield 410. The diaphragm 420 may be directly attached the storage bags 450 or engaged thereto via more tubing 430 or just have an exit for the milk where the milk flows from an exit tube 422 on the diaphragm 420 into the storage bag 450. The shield and bag assembly may include a harness to hold them around the user's neck or shoulders so they can be hands-free while pumping. The user as shown has also engaged the tubing 430 to the pump 110a.

The user may control the pump 110a using its screen or via their smart device 190. The pumping intensity, duration, and cycle length and other features may be controlled at either or both places as previously described. The user may use the table 140a top for their own laptop 195 or other uses.

The table 140a may contain storage shelves and a power outlets strip 170a. The storage unit 130a that may contain disposable supplies discussed herein like the tubing, diaphragms, and storage receptacles, and the refrigerator 120a may include storage hooks 132a thereon for clothing and bags as well as a mirror 134a.

In the kiosk 100 and pod 300, the system may have a seat 140 that is integral with or that is modular with the other components like the pump 110 and/or refrigerator and storage area. The modularity of all of the kiosk, pod, and cart allows for interchangeability of components if one breaks or requires service or upgrading. The seat 151 may also be a separate chair. The stations may also include a sink or wash station and a trash receptacle.

The kiosk 100 may include a privacy wall 160 that wraps around all or some sides of the seat 150 and pump 110, leaving at least an open entry point of access by the mother. The pod 300 may be fully enclosed by walls 360 and a door 362. It should be appreciated that both the kiosk 100 and pod 300 could be assembled in modular pieces for easy transport. The pieces could also be connectable to the other pieces like the pump 110, refrigerator 120, seat 150 etc.

Any of the cart or kiosk may include a privacy curtain for extra privacy in their more open environments.

The cart may include wheels so it can be moved or stored more easily and may be used in an embodiment shown in FIG. 2A with a storage unit 13a, refrigerator 120a, and pump 110a.

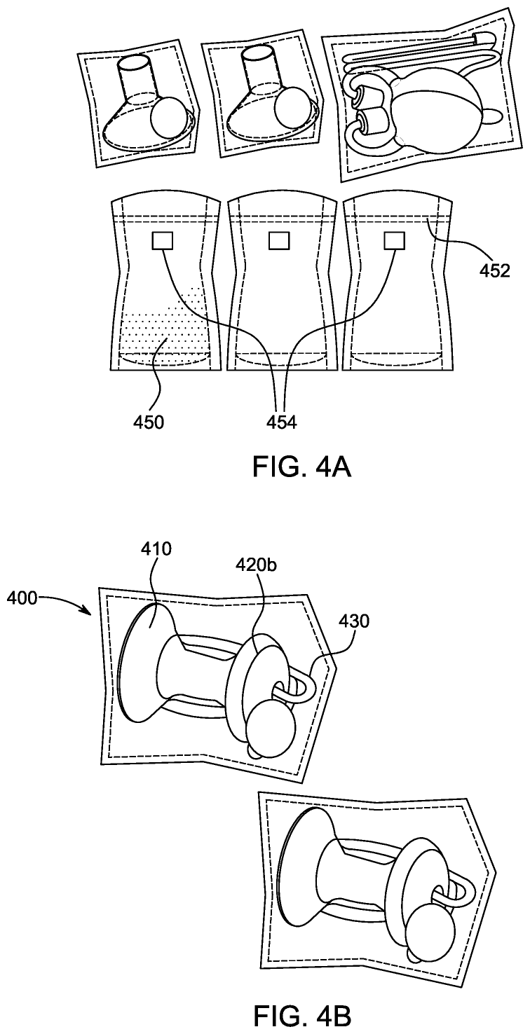

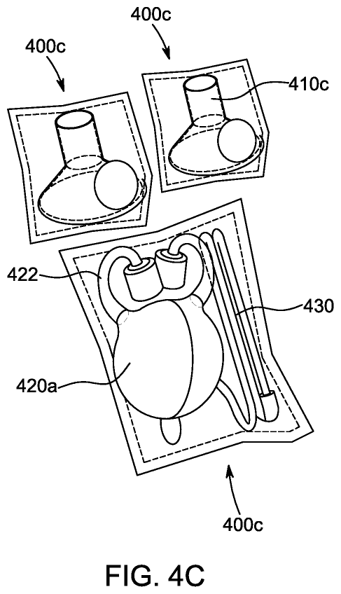

FIGS. 4A, 4B, and 4C show different bag, shield, and tube embodiments. In these, the separate components may be stored in a single package 400, 400c. Ideally but not necessarily, these packages may be relatively flat or at least squared off for efficient storage in the storage areas 130. Unlike traditional reusable components that require cleaning, these pieces may be disposable and/or recyclable, which allows for the mother on the go to pump and go—and maintain sterility.

The package 400 may include a breast shield 410 attached to a flattened diaphragm 420 that is itself attached to tubing 430. These pieces may all be integral with one another so that the only assembly the user has to do is to attach the tubes to a pump port. The package in FIG. 4B is similar but without the flattened diaphragm, having instead a slightly different shaped diaphragm 420b. The package 400c in FIG. 4C is different and may include separately packaged shields 410c from the diaphragm 420c. The diaphragm 420c in this embodiment is a single diaphragm 420c that attaches via exit tubes 422 to the shields 410c. The single diaphragm 420c attaches via tubing 430 to the pump 110 and may be engaged directly to storage bags 450 or to the bags 450 by further tubing 430. The package 400 may include one or more storage bags 450 that are configured to receive and store milk in an airtight container, preferably using a zip tight air seal 452. The storage bags 450 may have a tube port to receive tubing 430 that carries milk from the diaphragm 420.

FIGS. 5A-5E show different embodiments of the pump 110, 110a previously discussed. The pump 110a engages the table 140a and extends therefrom. A flange 510 acts as a seal between the pump 110a and the table 140a. The flange 510 may be of a flexible and washable material that may be easily replaced. As indicated by the arrow 505, the pump 110a may rotate relative to the table 140a in order that it may face the user if they are seated in the chair, standing, or seated in another chair—or even if the chair is located on the other side of the table 140a.

A top 520a of the pump 110a includes controls, which may be mechanical buttons or a touch face 525a. The controls may control pump speed, cycles, vacuum level, vacuum cycle speed, mode: expression or stimulation (a.k.a. let-down), cycle active or cycle paused. or other pump controls. The pump top 520a may include a near field communication NFC area 530a that may interface with a mobile device 190 or other interface. The pump 110a may include a mechanical tubing connector 550 shown in FIG. 5D.

The mechanical tubing connector 550 includes a tubing engagement portion 552 that engages the tubing 430 and a pump engagement portion 554 with a connection that may be tapered or even use a luer taper that engages an internal pump flange 114a. with both engagement portions connected, the pump 110a may draw air through the mechanical tubing connector 550, tubing 430, diaphragm 420, and breast shield 410. The connector 550 may include a key 555 that interacts with and allows the pump 110a to be activated. Without the key 555, the pump cannot be activated. The key 555 may be mechanical in nature to press a button or it may include a chip, magnet, or other proximity activator 560 that a sensor 570 senses, and when sensed, removes a lock on the pump 110a and allows it to be activated. The key 555 may be outside the pump 110a or inside the pump 110a.

FIG. 5E shows a mechanical embodiment of the key 555e, with components similar to the components in the key of FIG. 5D, with a mechanical tooth 560e that presses a switch 570e that activates the pump when engaged, or not when disengaged.

FIG. 5B shows another embodiment of the pump 110b. This pump 110b has a smooth and tapered top 520b that sheds liquids and is easy to clean, though the top 520b may also be angled. The flange 510b is squared in this embodiment to simplify milling in the table 140a. The pump 110b includes the connector port 570 as well.

FIG. 5C shows another embodiment of the pump 110c, with a similar flange 510c and a flush top 520c that is flush with the table 140a. There is, however, a raised top portion 525c that includes the connector port 570.

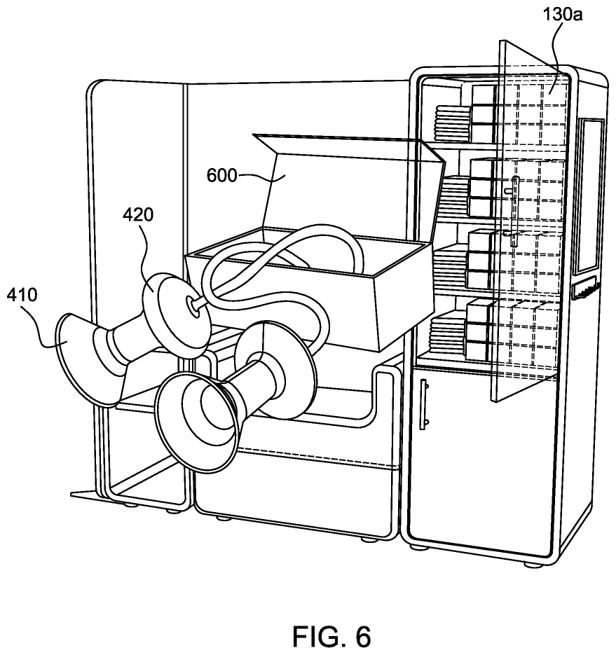

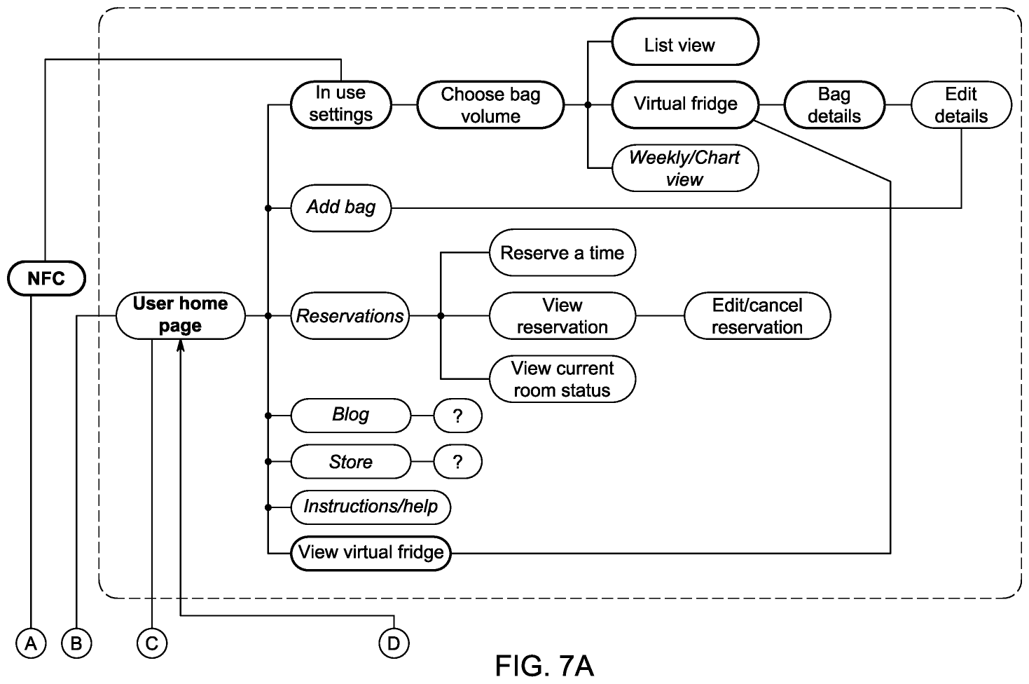

FIG. 6 shows an alternate storage package 600 within the storage area 130a.

It should be appreciated that milk storage bags may also be integral with these devices and include ports accessible to other tubing for clean transport and sealing of the milk obtained during pumping.

The Application

The mobile application mentioned above may control pump functions customized to a user and may provide these features:

(1) Location of open stations (with these stations using a location indicator such as GPS, IP addresses, or similar to show their locations on maps and other user detection means to show if they are free);

(2) inventory ordering for pods and/or users/owners;

(3) relevant targeted information and advertising; and

(4) user interactions and social networking and education.

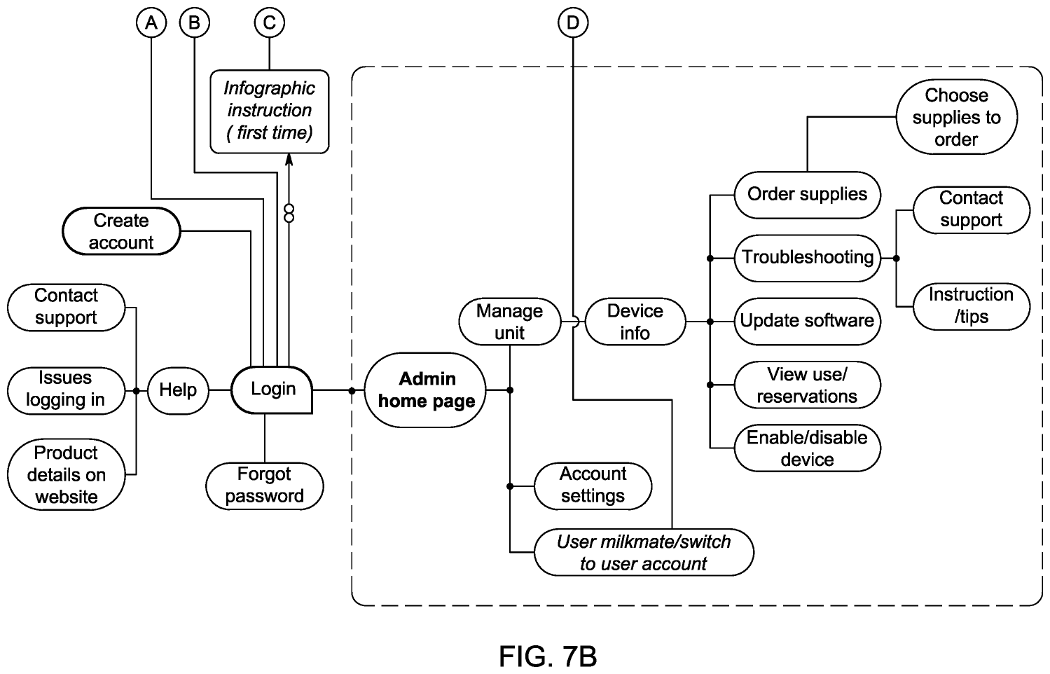

FIGS. 7A and 7B show the application's functionality in summary. The application allows for account creation. From a login screen, the user can contact support, report issues logging in, follow links to product details on the website, put in a forgotten password request, or request help (available at all times).

The login screen determines whether the user has user or admin privileges. The user privileges welcome the user to a home page after login. In use settings may include choosing the bag volume, managing a virtual refrigerator (a representation of a refrigerator that tracks the milk that has been pumped and stored), charts and reports regarding how much milk volume and how many days there have been pumped, and details for each bag and lists of same for current and historical bags pumped.

The user can add a new bag when pumping commences.

The user can also make and manage reservations.

The user can blog about their experience, make purchases from an online store, seek help with pumping, and view a virtual refrigerator.

The administration privileges allow a user to manage account settings and switch user accounts. The admin user can manage units (metric vs imperial), and manage device information for devices in the system. Such device information may include ordering supplies, troubleshooting and contacting support, updating software, view and edit reservations, and enable or disable devices.

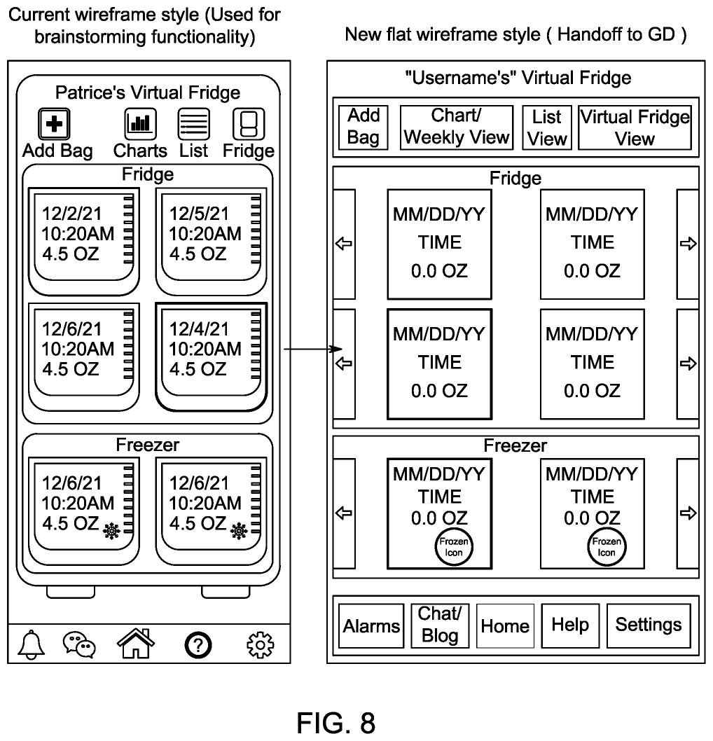

FIG. 8 shows a view of a virtual refrigerator that shows days, times, and amounts of milk pumped by a user. The milk can be in a virtual refrigerator or freezer, which represent a real refrigerator or freezer to help the user track the milk.

Additional System Features

Other features of the system shall be noted below.

The pump settings may auto reset between users so that subsequent users may use their own settings.

The pump display may display a personalized image to provide comfort and customization, for example, a mother's baby on the pump display and/or wall display.

The pump may interact with the user's mobile device and store the personalized pump setting preferences.

The mobile device and pump display may show settings including pump status, faults, diagnostics, and troubleshooting.

The mobile device and pump display may show personal pump usage tracking data and insights/recommendations.

The mobile device and pump display may show and communicate pump/kiosk busy signals and show pump available notifications so that waiting mothers may know when a pump is free to use.

The mobile device and pump display may allow a user to make reservations of the kiosks and pumps and the reservations may expire after a preset time if the user does not check in.

The system may track milk storage in refrigerator for expiration and send near expiration notifications to the user so they don't let it expire.

The system may show consumables kits availability, including availability by size for different sized consumables.

The mobile device and pump display may allow a user to request consumables kits for delivery either to the kiosk or directly to the user.

The system may track consumables used at a single location and provide the ability to automatically reorder consumables to maintain a preset level of stock based on usage patterns.

The system may send cleaning reminders (between pump uses) to either the user (for self-cleaning) or a designated party responsible for cleaning.

Consumable Features

Regarding the consumable, the following features may be found.

The breast shield may have various sizes.

The breast shield may be flexible to accommodate different sizes and may also include inserts that engage the mother's breast.

The mobile application may include a feature that measures the breast/nipple to indicate proper shield sizing.

The system may include a sizing template or tool that measures the breast/nipple to indicate proper shield sizing.

The system may include a single piece/fully assembled kit (shield, bag and tubing) for convenience.

The system may include a flat pack breast flange/shield (more compact for storage).

The system may include flat film tubing to allow for more compact packing.

The system may include peelable tubing (easier tube management).

The system may include insulated milk bags (to improve transit temp).

The system may include a wearable flange/shield/bag.

The system may include tubing that doubles as wearable support bra for pumping. Such tubing may hold the bags during pumping.

The system may include an integrated label printer for milk bag identification.

The system may include personalized consumables kits (QR coded, color coded).

The system may include sustainable and renewable features.

The system may include a consumables recycling service that includes a disposal container to collect consumables that may be recyclable.

The system may include a consumable's cleaning/sanitizing service for re-use. The service may be provided with the system and include human cleaning.

The system may include consumables cleaning/sanitizing feature in kiosk for re-use. This feature may include something like a machine washer specially shaped to wash the system components.

The system may include a bagless—pump that allows for milk to be directed into bottles.

The system may include a re-usable tubing (heavier duty, self-cleaning).

The system may include integrated consumables disposal in kiosk (for collection recycling or cleaning).

Other System Features

Several other features may be possible as well:

A swing-out/airline seat style table for laptop/worksurface may be available as part of the seat or refrigerator.

An easy mount pump that engages different types of furniture pieces.

The kiosk may include a wet sink.

The system (or pump itself) may include an auto pump shut-off to prevent overfilling.

The system may include a consumables vending machine or a machine with only smart lock access.

The system may include milk bag storage organization inserts for the refrigerator (customizing a standard refrigerator interior).

The system may include a white noise generator/noise reduction to give the user privacy.

The system may include a backup battery so that pumping may continue in the absence of power.

While the invention has been described with reference to the embodiments above, a person of ordinary skill in the art would understand that various changes or modifications may be made thereto without departing from the scope of the claims.

1. A modular multiuser breast pumping station that includes modular components comprising:
a multiuser breast pump that engages tubing that is engaged to breast shields, wherein the breast shields are engaged to at least one storage bag; and
a storage area for the tubing, breast shields, and storage bags for milk;
wherein when the breast shield is attached to a user's breast, the multiuser breast pump draws a vacuum through the tubing and breast shield, thereby drawing milk from the user's breast that is directed into the storage bags.

2. The station of claim 1, further comprising a refrigerator configured to store the storage bags containing milk obtained from a user's breast.

3. The station of claim 1, further comprising privacy walls that protect the user from view.

4. The station of claim 1, further comprising a privacy curtain that protects the user from view.

5. The station of claim 1, wherein the pump is controlled by a mobile device.

6. The station of claim 5, wherein the control is done using an application on the mobile device.

7. The station of claim 6, further comprising a location indication that indicates the station's location to the application.

8. The station of claim 5, wherein the station detects a user's presence and communicates that presence to other users.

9. The station of claim 1, further comprising a table, wherein the pump is engaged to the table.

10. The station of claim 9, further comprising a flange that engages the pump to the table.

11. The station of claim 1, wherein the pump includes settings for at least one of vacuum strength, cycling, cycling strength, vacuum release, duration, vacuum level, vacuum cycle speed, mode: expression or stimulation, cycle active or cycle paused that a user may set and store or that may be accessible and operable through a mobile app that connects to the pump.

12. The station of claim 1, wherein the pump comprising a touch screen top.

13. The station of claim 1, wherein the pump has a top that is flush to a table in which it is mounted.

14. The station of claim 1, wherein the pump engages the tubing through a key, wherein the pump is inoperable in the absence of the key.

15. The station of claim 14, wherein the pump comprises an activation switch that the key engages.
